(19) United States
(12) Patent Application Publication
(10) Pub. No.: US 2024/0100313 A1
(43) Pub. Date: Mar. 28, 2024

(54) COUPLING ARRANGEMENT FOR AIR DELIVERY CIRCUITS

(71) Applicant: KONINKLIJKE PHILIPS N.V., Eindhoven (NL)

(72) Inventors: RICHARD THOMAS HAIBACH, VERONA, PA (US); LAUREN PATRICIA CHODKOWSKI, PITTSBURGH, PA (US); STEVEN WILLIAM PEPIN, KIRKLAND, WA (US)

(21) Appl. No.: 18/371,737

(22) Filed: Sep. 22, 2023

Related U.S. Application Data
(60) Provisional application No. 63/410,332, filed on Sep. 27, 2022.

(51) Int. Cl. A61M 39/10

(52) U.S. Cl. CPC A61M 39/10; A61M 2039/1016

(57) ABSTRACT

A coupling arrangement for use in an air delivery circuit includes a first connector tube defining a first passage therethrough and a second connector tube defining a second passage therethrough. The second connector tube telescopically receives a portion of the first connector tube within the second passage. A release mechanism includes wedges coupled to the first or second connector tube. The first and second connector tubes have a number of corresponding features that latch the first connector tube within the second passage of the second connector tube when the first connector tube is axially inserted along the longitudinal axis a predetermined distance. Actuation of the wedges radially toward the longitudinal axis causes axial movement among the first connector tube and the second connector tube a second predetermined distance that is sufficient to unlatch the corresponding features.

### CROSS-REFERENCE TO RELATED APPLICATIONS

This patent application claims the priority benefit under 35 U.S.C. § 119(e) of U.S. Provisional Application No. 63/410,332, filed on Sep. 27, 2022, the contents of which are herein incorporated by reference.

### BACKGROUND OF THE INVENTION

### 1. Field of the Invention

The present invention relates to arrangements for communicating a flow of a treatment gas to the airway of a patient and, more particularly, to coupling arrangements for use therein. The present invention further relates to methods of operating such coupling arrangements.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces a ventilator or pressure support device with the airway of the patient, so that a flow of breathing gas can be delivered from a pressure/flow generating device to the airway of the patient. It is known to maintain such devices on the face of a wearer by a headgear having one or more straps adapted to fit over/around the patient's head. Because such patient interface devices are typically worn for an extended period of time, it is important for the headgear to maintain the mask component of the device in a tight enough seal against the patient's face without discomfort.

Conventional CPAP masks commonly utilize coupling arrangements for coupling supply conduits to masks and/or to other components that prevent undesired axial displacement and uncoupling of the components. Such arrangements present challenges to some users which may not be able to exert the physical force required to perform coupling and decoupling operations thereof. Further, some users may lack the dexterity required to perform multistage coupling and decoupling operations required by such arrangements. There is therefore a need to provide improved coupling arrangements for use with CPAP masks and related components.

### SUMMARY OF THE INVENTION

Accordingly, it is an object of the present invention to provide a coupling arrangement for use in an air delivery circuit. The coupling arrangement comprises: a first connector tube defining a first passage therethrough; a second connector tube defining a second passage therethrough disposed about a longitudinal axis, the second connector tube structured to telescopically receive a portion of the first connector tube within the second passage of the second connector tube; and a release mechanism having a number of actuatable wedges, the release mechanism coupled to one of the first connector tube or the second connector tube, wherein the first connector tube and the second connector tube each have a number of corresponding features which latch the first connector tube within the second passage of the second connector tube when the first connector tube is axially inserted along the longitudinal axis a predetermined distance within the second passage of the second connector tube, and wherein actuation of the number of wedges radially toward the longitudinal axis causes axial movement among the first connector tube and the second connector tube a second predetermined distance sufficient to unlatch the corresponding features.

The number of actuatable wedges may comprise at least two wedges positioned as an opposing pair. The release mechanism may comprise a biasing member coupled between the at least two wedges, the biasing member being structured to bias the at least two wedges apart.

Each wedge of the number of actuatable wedges may comprise a wedge body having a radially inward-facing sloped surface structured to engage one of the first connector tube or the second connector tube, an outward facing pressing surface, a base surface structured to engage the other one of the first connector tube and the second connector tube, and a leading edge disposed between the inward-facing sloped surface and the base surface that is structured to engage an intersection between the first connector tube and the second connector tube. The pressing surface of each wedge body may comprise a textured arrangement. The sloped surface may be disposed at an acute angle relative to the base surface.

The number of corresponding features of the first connector tube may comprise at least one prong, and the number of corresponding features of the second connector tube may comprise a shoulder portion bounding the second passage. A free end of each prong may be hooked onto the shoulder portion when the second connector tube is coupled to the first connector tube, and actuating the release mechanism may force the prong to be unhooked from the shoulder and decouples the second connector tube from the first connector tube.

The one of the first connector tube or the second connector tube may extend from a manifold portion of a tubing assembly, and the other of the first connector tube or the second connector tube may extend from a flexible conduit.

The one of the first connector tube or the second connector tube may extend from an elbow connector, and the other of the first connector tube or the second connector tube may extend from a faceplate.

As another aspect of the present invention a respiratory interface system for use in providing a regimen of respiratory therapy to a patient is provided. The respiratory interface system comprises: a pressure generating device; a delivery conduit; a patient interface; and a coupling arrangement as previously described coupling the pressure generating device to the delivery conduit or the delivery conduit to the patient interface.

The coupling arrangement may couple the delivery conduit to the patient interface, the one of the first connector tube or the second connector tube may extend from a manifold portion of a tubing assembly associated with the patient interface, and the other of the first connector tube or the second connector tube may extend from the delivery conduit.

The coupling arrangement may couple the delivery conduit to the patient interface, the one of the first connector tube or the second connector tube may extend from an elbow connector coupled to the delivery conduit, and the other of the first connector tube or the second connector tube may extend from a faceplate associated with the patient interface.

As yet a further aspect of the present invention a method of operating a coupling arrangement is provided. The method comprises: coupling a first connector tube and second connector tube by inserting the first connector tube into the second connector tube a distance along a longitudinal axis of the second connector tube such that a number of corresponding features of the first connector tube and the second connector tube latch the first connector tube within the second passage of the second connector tube; and decoupling the first connector tube and second connector tube by actuating a number of wedges of a release mechanism radially toward the longitudinal axis by grasping/pressing the number of wedges with a single hand thus causing axial movement among the first connector tube and the second connector tube a second predetermined distance that is sufficient to unlatch the first connector tube from the second connector tube.

These and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a partially schematic view of a respiratory interface system in accordance with one example embodiment of the present invention;

FIG. 2 is a perspective view of a portion of the respiratory interface system of FIG. 1;

FIG. 3 is a sectional view of an example coupling arrangement of the system of FIG. 1 shown in a coupled positioning with a release mechanism thereof shown unactuated;

FIG. 4 is a sectional view of the coupling arrangement of FIG. 3 in a coupled positioning with the release mechanism thereof shown starting to be actuated;

FIG. 5 is a sectional view of the coupling arrangement of FIGS. 3 and 4 shown in an uncoupled positioning with the release mechanism thereof fully actuated;

FIG. 6 is a detail view of a portion of the sectional view of FIG. 3;

FIG. 7 is a perspective view of the release mechanism of FIGS. 1-6;

FIG. 8 is a partially schematic view of a portion of a respiratory interface system including a coupling arrangement in accordance with another example embodiment of the present invention;

FIG. 9 is a partially exploded view of components of the coupling arrangement of FIG. 8;

FIG. 10 is a perspective view of one of the components of the coupling arrangement of FIGS. 8 and 9;

FIG. 11 is a perspective view of the release mechanism of the coupling arrangement of FIGS. 8-10; and

FIG. 12 is a sectional view of the coupling arrangement of FIG. 12 taken along line 12-12 of FIG. 8.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the statement that two or more parts or components “engage” one another shall means that the parts exert a force against one another either directly (i.e., “directly engage”) or through one or more intermediate parts or components. As used herein, the word “unitary” means a component is created as a single piece or unit. That is, a component that includes pieces that are created separately and then coupled together as a unit is not a “unitary” component or body. As used herein, the term “number” shall mean one or an integer greater than one (i.e., a plurality).

As used herein, a “coupling assembly” includes two or more couplings or coupling components. The components of a coupling or coupling assembly are generally not part of the same element or other component. As such the components of a “coupling assembly” may not be described at the same time in the following description.

As used herein, a “coupling” is one element of a coupling assembly. That is, a coupling assembly includes at least two components, or coupling components, that are structured to be coupled together. It is understood that the elements of a coupling assembly are compatible with each other. For example, in a coupling assembly, if one coupling element is a snap socket, the other coupling element is a snap plug.

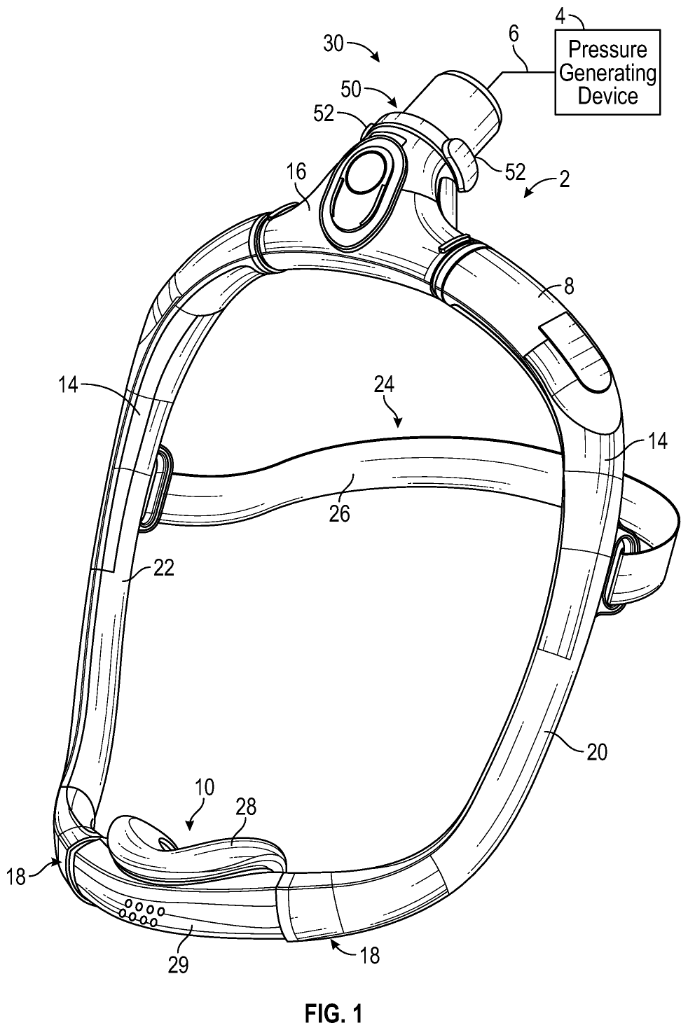

Referring to FIG. 1, a respiratory interface system 2 for use in providing a regimen of respiratory therapy to a patient (not shown) according to one exemplary embodiment of the present invention is shown. Respiratory interface system 2 includes a pressure generating device 4 (shown schematically), a delivery conduit 6 (shown schematically) coupled between pressure generating device and a tubing assembly 8, and a patient interface 10 coupled to tubing assembly 8. Pressure generating device 4 is structured to generate a flow of positive pressure breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, PA), and auto-titration pressure support devices. Delivery conduit 6 is structured to communicate the flow of breathing gas from pressure generating device 4 to patient interface device 10 through tubing assembly 8 (the flow of breathing gas enters tubing assembly 8 at the top of the head of a patient when tubing assembly 8 and patient interface device 10 are positioned on the head of the patient). Delivery conduit 6, tubing assembly 8 and patient interface device 10 are often collectively referred to as a patient circuit.

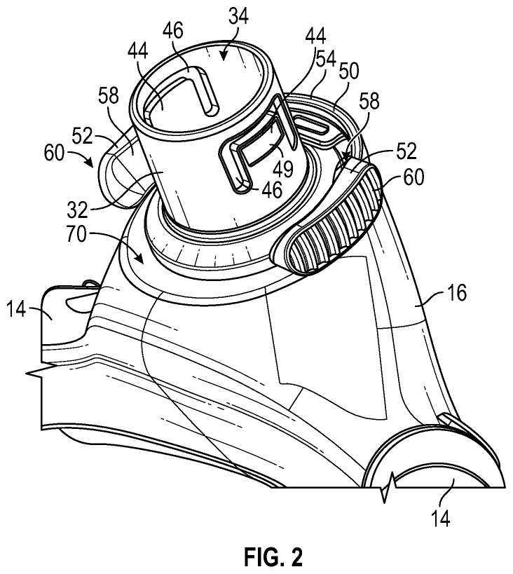

Continuing to refer to FIG. 1, and additionally to FIG. 2, tubing assembly 8 includes a number of tubular portions 14 which each extend from a common manifold portion 16 to a respective distal end 18. Distal end 18 of each tubular portion 14 is coupled to patient interface 10. In exemplary embodiments, tubing assembly 8 is made from plastic and/or silicone and may be formed as a single unitary member or alternately may be formed from a number of separately formed components that are then coupled together via a suitable process. Tubing assembly 8 and/or patent interface 10 may also be formed from other suitable materials (e.g., fabric) without varying from the scope of the present invention.

Manifold portion 16 is structured to be coupled to delivery conduit 6 via an arrangement discussed in detail below. When tubing assembly 8 is disposed on the head of a patient, manifold portion 16 is disposed generally at the top of the head of the patient and tubular portions 14 each extend generally downward from manifold portion 16 to patient interface device 10. In the exemplary embodiment shown in FIGS. 1 and 2, tubing assembly 8 includes two tubular portions 14, also referred to herein as left side arm 20 and right side arm 22, which each have a generally non-circular cross-section. That is, each tubular portion 14 is not substantially circular. In another exemplary embodiment, not shown, tubing assembly 8 includes a single tubular portion 14 that extends centrally, i.e., from manifold portion 16 generally over the patient's forehead and nose, to patient interface 10.

In an exemplary embodiment, each tubular portion 14 has a generally D-shaped cross-section wherein the generally flat side of the D-shape is disposed adjacent the patient's head while the curved portion faces away from the patient's head. In other example embodiment, each tubular portion 14 has a generally oval shaped cross-section. Tubular portions 14, i.e., left and right side arms 20, 22, in conjunction with manifold portion 16, encircle, or partially encircle, the head of a patient when positioned thereon for delivering a regimen of respiratory therapy to a patient such as previously discussed. Accordingly, it is to be appreciated that tubing assembly 8, as a result of its basic structure and positioning, generally functions not only as a portion of the supply conduit for providing gas to/from patient interface 10, but also generally functions as a frame, securing patient interface 10 to the head of a patient. Further, in order to help secure patient interface device 10 and tubing assembly 8 to the head of a patient, tubing assembly 8 may further include a support assembly 24, which in the example embodiment shown in FIG. 1 is a strap member 26 coupled to left and right-side arms 20, 22 which partially encircles, the head of a patient. That is, strap member 26 is structured to engage the rear of the head of a patient, and in an exemplary embodiment of the present invention is structured to fit generally just below the occipital bone of a patient.

Continuing to refer to FIG. 1, patient interface 10 includes a sealing arrangement 28 that is structured to seal generally about an airway of a patient. In an exemplary embodiment, such as illustrated in FIG. 1, sealing arrangement 28 is a nasal cushion made of a soft, flexible material, such as, without limitation, silicone, an appropriately soft thermoplastic elastomer, a closed-cell foam, or any other suitable material or combination of such materials, however, it is to be appreciated that any type of sealing arrangement, such as a nasal/oral mask, a nasal pillow or a full face mask, which facilitates the delivery of the flow of breathing gas to the airway of a patient, may be used as sealing arrangement 28 while remaining within the scope of the present invention. Sealing arrangement 28 is positioned about a patient aperture (not numbered) which provides access to a main cavity (not numbered) defined within a body 29 of patient interface 10. The main cavity is structured to receive the flow of breathing gas produced by pressure generating device 4 (and conveyed to the main cavity via conduit 6 and tubular portions 14 of tubing assembly 8) which is then conveyed to the airway of a patient through the patient aperture. In the example embodiment illustrated in FIG. 1, the main cavity receives the flow of breathing gas produced by pressure generating device 4 via a pair of inlet ports (not numbered) positioned at either end of patient interface 10 that are each coupled to a respective distal end 18 of left and right-side arms 20, 22.

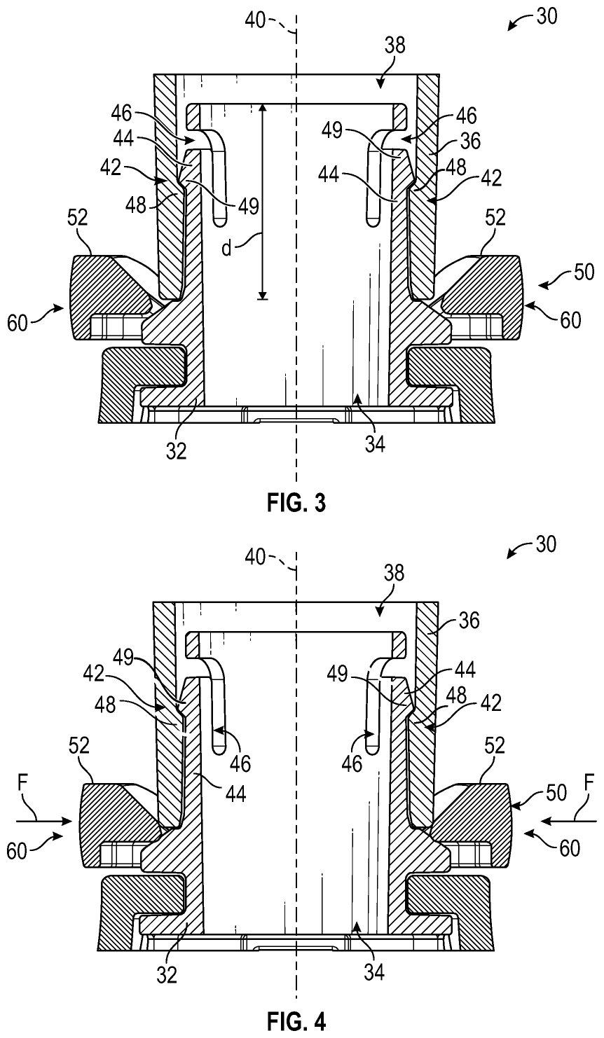

Respiratory interface system 2 further includes at least one coupling arrangement 30 that is structured to provide a detachable connection between conduit 6 and manifold portion 16. Referring to the sectional views of FIGS. 3-5, coupling arrangement 30 comprises a first connector tube 32 defining a first passage 34 therethrough and a second connector tube 36 defining a second passage 38 therethrough and disposed about a longitudinal axis 40. Second connector tube 36 is structured to telescopically receive a portion of first connector tube 32 within second passage 38 thereof. First connector tube 32 and second connector tube 36 have a number of corresponding features 42 which latch first connector tube 32 within second passage 38 of second connector tube 36 when first connector tube 32 is axially inserted along longitudinal axis 40 a predetermined distance d (FIG. 3) within second passage 38 such that first connector tube 32 is axially constrained within second passage 38 of second connector tube 36 in a manner such that first and second connector tubes 32 and 36 do not inadvertently become disconnected.

In the example embodiment shown in FIGS. 2-6, the number of corresponding features 42 comprises a number of prongs 44 provided as a portion of first connector tube 32. Each prong 44 extends generally parallel with respect to longitudinal axis 40 and is delineated by a corresponding relief hole 46 extending radially through first connector tube 32. In such example, the number of corresponding features 42 further includes a number of shoulder portions 48 defined in second connector tube 36 bounding second passage 38. As shown in FIGS. 3, 4 and 6, in a coupled positioning of first and second connector tubes 32, 36 (i.e., when first connector tube 32 is inserted the predetermined distance d into second connector tube 36 such as previously discussed), a free end 49 of each prong 44 is engaged with (e.g., hooked onto) a corresponding one of the number of shoulder portions 48 (or a corresponding portion of a single shoulder portion 48 extending circumferentially about the interior of second connector tube 36).

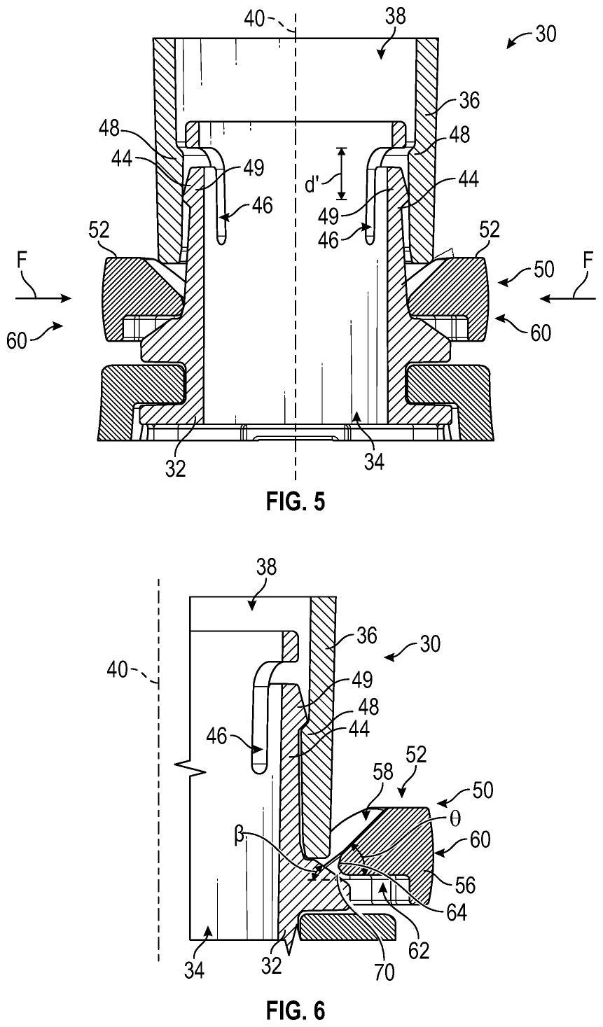

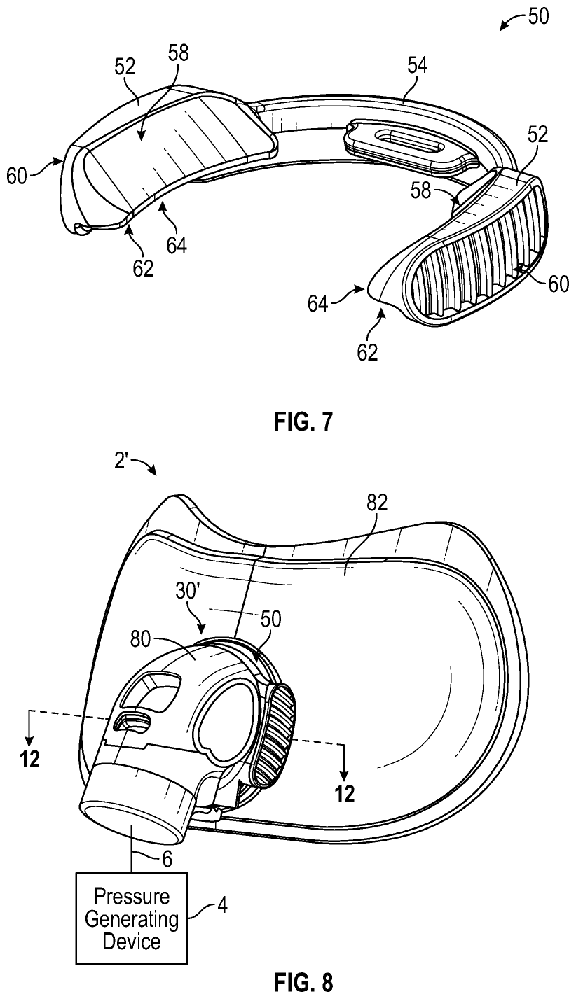

Continuing to refer to FIGS. 2-6, and additionally FIG. 7, coupling arrangement 30 further comprises a release mechanism 50 having a number of actuatable wedges 52 (two are shown in the example embodiment of FIGS. 2-5 and 7) and a number of biasing members 54 (one is shown in the FIGS. 3-5 and 7). Release mechanism 50 may be coupled to one of first connector tube 32 or second connector tube 36. In the example shown in FIG. 2, release mechanism 50 is coupled to first connector tube 32. As shown in the progressive sectional views of FIGS. 3-5, actuation of the number of wedges 52 radially inward toward longitudinal axis 40 (e.g., without limitation, such as by pressing together using a thumb and index finger of a single hand of a user) causes a corresponding separating axial movement along longitudinal axis 40 among first connector tube 32 and second connector tube 36 a second predetermined distance d′ (FIG. 5) that is sufficient to unlatch first connector tube 32 from second connector tube 36 (i.e., to disengage corresponding features 42). In the example embodiment shown in FIGS. 3-7, the number of actuatable wedges 52 comprises two wedges 52 positioned as a generally opposing pair that are biased apart (i.e., away from longitudinal axis 40) by biasing member 54. Actuating release mechanism 50 forces axial movement among first connector tube 32 and second connector tube 36 that forces each prong 44 to become unhooked from its corresponding shoulder portion 48 and thus decouples first connector tube 32 and second connector tube 36.

Continuing to refer to FIGS. 6 and 7, each wedge 52 of the number of actuatable wedges 52 of release mechanism 50 comprises a wedge body 56 that includes a radially inward facing (i.e., toward longitudinal axis 40) sloped surface 58, an outward facing pressing surface 60, a base surface 62, and a leading edge 64. In the example embodiment shown in FIGS. 6 and 7, base surface 62 is positioned generally perpendicular to longitudinal axis 40 while pressing surface 60 is positioned generally parallel to longitudinal axis 40. Sloped surface 58 is disposed at an acute angle θ (FIG. 6, e.g., 45° or any other suitable acute angle) relative to base surface 62. In the example embodiment shown in FIGS. 2-7, sloped surface 58 is both angled and generally curves around longitudinal axis 40 and thus is formed generally as a portion of an inner conical surface (i.e., inside surface of a cone). Leading edge 64 is disposed opposite pressing surface 60, generally at the intersection of sloped surface 58 and base surface 62.

As shown in the sectional views of FIGS. 3-5, leading edge 64 is positioned/structured to engage an outer intersection between first connector tube 32 and second connector tube 36 when an external force F is applied to pressing surface 60, thus causing actuatable wedge 52 to generally force (i.e., “wedge”) first connector tube 32 and second connector tube 36 apart so as to move axially along longitudinal axis 40 in opposite directions, thus unlatching corresponding features 42 and uncoupling first and second connector tubes 32 and 36. As shown in the example of FIGS. 2 and 7, pressing surface 60 may be provided with a textured arrangement (e.g., grooves, ridges, or any other suitable arrangement) to resist slippage when such surface is engaged by the fingers of a patient or other person(s) desiring to uncouple connector tubes 32 and 36.

As shown in the example embodiment of FIGS. 2-6, in order to assist in the axial separation/displacement of connector tubes 32 and 36, first connector tube 32 includes a radially outward facing sloped surface 70 that is positioned at an angle β (FIG. 6) relative to a reference line disposed perpendicular to longitudinal axis 40. In the example shown, sloped surface 70 is positioned at an angle β of about 30°, however, sloped surface 70 is generally sloped to provide a clearance for wedge(s) 52 to readily interact with the intersection between first and second connector tubes 32 and 36 and thus angle β may be varied as needed for a particular application. Similar to sloped surface 58 of each wedge 52, sloped surface 70 curves around longitudinal axis 40 and thus is formed generally as a portion of an outer conical surface (i.e., outside surface of a cone). As shown in the sequential sectional views of FIGS. 3-5, sloped surface 70 is disposed at the intersection of tube portions 32 and 36 and thus interacts with leading surface 64 of each wedge 52 to assist in the axial separation/displacement of connector tubes 32 and 36 provided by the interaction therewith by each wedge 52.

While the example embodiment described in conjunction with FIGS. 1-7 shows an example coupling arrangement 30 in which first connector tube 32 extends from manifold portion 16 of tubing assembly 8, it is to be appreciated that the arrangement of the components of coupling arrangement 30 can be generally reversed (i.e., second connector tube 36 extending from manifold portion 16) without varying from the scope of the present invention. Further, while the example embodiment described in conjunction with FIGS. 1-7 shows an arrangement wherein coupling arrangement 30 couples conduit 6 to manifold portion 16 of tubing assembly 8, it is to be appreciated that such coupling arrangement can be employed at one or more other locations along the pathway of the flow of breathing gas produced by pressure generating device 4 without varying from the scope of the present invention and/or may be used to couple various other devices beyond those shown in FIG. 1.

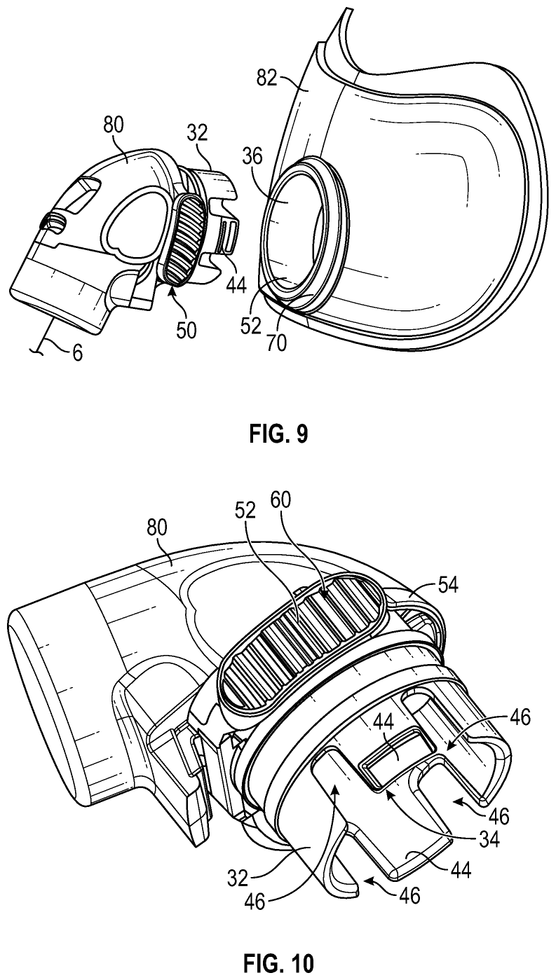

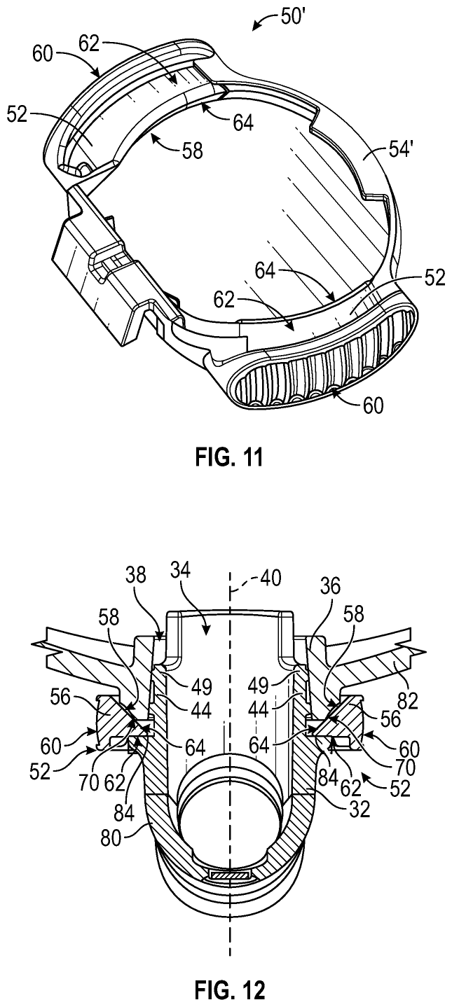

Referring now to FIGS. 8-12, another example embodiment of a coupling arrangement 30′ in accordance with the present invention will now be described. Coupling arrangement 30′ includes similar elements as coupling arrangement 30 previously discussed with a few distinctions. In coupling arrangement 30′ first connector tube 32 is formed as a portion of an elbow connector 80 that is coupled to, and extends from conduit 6 (shown schematically in FIGS. 8 and 9), while second connector tube 36 is formed as a portion of a faceplate 82 for use in a patient interface. Additionally, base surface 62 of each wedge member 52 of release mechanism 50 engages with a corresponding surface 84 (FIG. 12) of first connector tube 32 while sloped surface 58 of each wedge member 52 interacts with a portion 70 (FIGS. 9 and 12) of second connector tube 36 (i.e., faceplate 82).

While the example arrangement shown in FIGS. 8-12 shows a coupling arrangement 30′ in which first connector tube 32 extends from elbow connector 80 and second connector tube 36 extends from faceplate 82, it is to be appreciated that the arrangement of the components of coupling arrangement 30′ can be generally reversed (i.e., first connector tube 32 extending from faceplate 82 and second connector tube 36 extending from elbow connector 80) without varying from the scope of the present invention.

From the foregoing examples it is to be appreciated that in general a method of operating either of coupling arrangement 30 and/or 30′ includes coupling first connector tube 32 and second connector tube 36 by inserting first connector tube 32 into second connector tube 36 the distance d along longitudinal axis 40 such that corresponding features 42 of first and second connector tubes 32 and 36 latch first connector tube 32 within second passage 38 of second connector tube 36. Further, such method may include decoupling first connector tube 32 and second connector tube 36 by actuating the number of wedges 52 of release mechanism 50 radially toward longitudinal axis 40 (e.g., by grasping/pressing wedges 52 toward each other with a single hand), thus causing axial movement among first connector tube 32 and second connector tube 36 a second predetermined distance d′ that is sufficient to unlatch first connector tube 32 from second connector tube 36.

From the foregoing it is thus to be appreciated that embodiments of the present invention provide a means for selectively coupling components of an air delivery circuit used with a CPAP machine or other air delivery arrangement. Embodiments of the present invention further provide a means to repeatedly couple and decouple components in a manner which requires less force and/or motions than conventional arrangements and provides for one-handed uncoupling.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A coupling arrangement for use in an air delivery circuit, the coupling arrangement comprising:
a first connector tube defining a first passage therethrough;
a second connector tube defining a second passage therethrough disposed about a longitudinal axis, the second connector tube structured to telescopically receive a portion of the first connector tube within the second passage of the second connector tube; and
a release mechanism having a number of actuatable wedges, the release mechanism coupled to one of the first connector tube or the second connector tube,
wherein the first connector tube and the second connector tube each have a number of corresponding features which latch the first connector tube within the second passage of the second connector tube when the first connector tube is axially inserted along the longitudinal axis a predetermined distance (d) within the second passage of the second connector tube, and
wherein actuation of the number of wedges radially toward the longitudinal axis causes axial movement among the first connector tube and the second connector tube a second predetermined distance (d′) sufficient to unlatch the corresponding features.

2. The coupling arrangement of claim 1, wherein the number of actuatable wedges comprises at least two wedges positioned as an opposing pair.

3. The coupling arrangement of claim 2, wherein the release mechanism comprises a biasing member coupled between the at least two wedges, the biasing member being structured to bias the at least two wedges apart.

4. The coupling arrangement of claim 1, wherein each wedge of the number of actuatable wedges comprises a wedge body having a radially inward-facing sloped surface structured to engage one of the first connector tube or the second connector tube, an outward facing pressing surface, a base surface structured to engage the other one of the first connector tube and the second connector tube, and a leading edge disposed between the inward-facing sloped surface and the base surface that is structured to engage an intersection between the first connector tube and the second connector tube.

5. The coupling arrangement of claim 4, wherein the pressing surface of each wedge body comprises a textured arrangement.

6. The coupling arrangement of claim 4, wherein the sloped surface is disposed at an acute angle θ relative to the base surface.

7. The coupling arrangement of claim 1, wherein the number of corresponding features of the first connector tube comprises at least one prong, and wherein the number of corresponding features of the second connector tube comprises a shoulder portion bounding the second passage.

8. The coupling arrangement of claim 7, wherein a free end of each prong is hooked onto the shoulder portion when the second connector tube is coupled to the first connector tube, and wherein actuating the release mechanism forces the prong to be unhooked from the shoulder and decouples the second connector tube from the first connector tube.

9. The coupling arrangement of claim 1, wherein the one of the first connector tube or the second connector tube extends from a manifold portion of a tubing assembly, and the other of the first connector tube or the second connector tube extends from a flexible conduit.

10. The coupling arrangement of claim 1, wherein the one of the first connector tube or the second connector tube extends from an elbow connector, and the other of the first connector tube or the second connector tube extends from a faceplate.

11. A respiratory interface system for use in providing a regimen of respiratory therapy to a patient, the respiratory interface system comprising:
a pressure generating device;
a delivery conduit;
a patient interface; and
a coupling arrangement coupling the pressure generating device to the delivery conduit or the delivery conduit to the patient interface, the coupling arrangement comprising:
a first connector tube defining a first passage therethrough;
a second connector tube defining a second passage therethrough disposed about a longitudinal axis, the second connector tube structured to telescopically receive a portion of the first connector tube within the second passage of the second connector tube; and
a release mechanism having a number of actuatable wedges, the release mechanism coupled to one of the first connector tube or the second connector tube,
wherein the first connector tube and the second connector tube each have a number of corresponding features which latch the first connector tube within the second passage of the second connector tube when the first connector tube is axially inserted along the longitudinal axis a predetermined distance (d) within the second passage of the second connector tube, and
wherein actuation of the number of wedges radially toward the longitudinal axis causes axial movement among the first connector tube and the second connector tube a second predetermined distance (d′) sufficient to unlatch the corresponding features.

12. The respiratory interface system of claim 11, wherein the number of actuatable wedges comprises at least two wedges positioned as an opposing pair, wherein the release mechanism comprises a biasing member coupled between the at least two wedges, and wherein the biasing member is structured to bias the at least two wedges apart.

13. The respiratory interface system of claim 11, wherein: the coupling arrangement couples the delivery conduit to the patient interface, the one of the first connector tube or the second connector tube extends from a manifold portion of a tubing assembly associated with the patient interface, and the other of the first connector tube or the second connector tube extends from the delivery conduit.

14. The respiratory interface system of claim 11, wherein the coupling arrangement couples the delivery conduit to the patient interface, the one of the first connector tube or the second connector tube extends from an elbow connector coupled to the delivery conduit, and the other of the first connector tube or the second connector tube extends from a faceplate associated with the patient interface.

15. A method of operating a coupling arrangement, the method comprising:
coupling a first connector tube and second connector tube by inserting the first connector tube into the second connector tube a distance (d) along a longitudinal axis of the second connector tube such that a number of corresponding features of the first connector tube and the second connector tube latch the first connector tube within the second passage of the second connector tube; and
decoupling the first connector tube and second connector tube by actuating a number of wedges of a release mechanism radially toward the longitudinal axis by grasping/pressing the number of wedges with a single hand thus causing axial movement among the first connector tube and the second connector tube a second predetermined distance (d′) that is sufficient to unlatch the first connector tube from the second connector tube.
